(19) United States
(12) Patent Application Publication
(10) Pub. No.: US 2017/0035341 A1
(43) Pub. Date: Feb. 9, 2017

(54) SMART DEVICE FOR BLADDER MAPPING

(71) Applicant: Boston Scientific Scimed, Inc., Maple Grove, MN (US)

(72) Inventors: Sandra Nagale, Bolton, MA (US); Shibaji Shome, Arden Hills, MN (US); Timothy Harrah, Cambridge, MA (US); Mark Boden, Harrisville, RI (US); Allan Shuros, St. Paul, MN (US); Bryan Clark, Forest Lake, MN (US); Amedeo Chiavetta, Derry, NH (US); Lynne Swanson, Edina, MN (US); Dennis Werner, Big Lake, MN (US)

(21) Appl. No.: 15/228,552

(22) Filed: Aug. 4, 2016

Related U.S. Application Data
(60) Provisional application No. 62/201,308, filed on Aug. 5, 2015.

(51) Int. Cl. A61B 5/20; A61B 1/07; A61B 1/04; A61B 18/14; A61B 1/00; A61B 5/0488; A61N 1/36; A61B 5/00; A61B 1/307

(52) U.S. Cl. CPC A61B 5/202; A61B 5/6858; A61B 5/6885; A61B 5/74; A61B 1/07; A61B 1/04; A61B 1/307; A61B 1/00045; A61B 5/04882; A61B 5/4836; A61B 5/6853; A61N 1/36007; A61B 18/1492; A61B 2562/166; A61B 2018/00577

(57) ABSTRACT

Systems, devices and methods for the treatment of bladder conditions using bladder visualization without the need for optical elements and for subsequent direct electrical pacing are provided. The systems, devices and methods generally apply pacing stimulus directly to the bladder wall, from one or more of the inner and outer bladder surfaces.

### CROSS-REFERENCE TO RELATED APPLICATIONS

This application claims priority to U.S. Provisional Application Ser. No. 62/201,308, filed on Aug. 5, 2015, the entire disclosure of which is herein incorporated by reference.

### FIELD OF THE DISCLOSURE

This application relates to the field of medical devices and medical procedures. More particularly, the application is related to devices and methods for noninvasive electrophysiological treatment, for example of urological conditions.

### BACKGROUND

The urinary bladder is a hollow, elastic organ that collects urine produced by the kidneys prior to urination (also referred to as “voiding” or “micturition”). The wall of the bladder generally includes an inner mucosal layer, a submucosal layer, and a muscular layer comprising, from inside-out, inner longitudinal, circular and outer longitudinal sublayers. Over the muscular layer are one or more connective tissue layers referred to as the serosa and adventitia. Between the bladder and the urethra is at least one bladder sphincter (the external bladder sphincter) that regulates the flow of urine from the bladder into the urethra during urination.

Contraction and relaxation of the bladder sphincter(s), and contraction of the bladder wall (also referred to as the “detrusor muscle”) are controlled by both somatic and autonomic nervous systems and, on the autonomic side, by both the sympathetic and parasympathetic nervous systems. Sensory information from stretch receptors within the muscular layer of the bladder is conveyed by sensory afferents extending from the bladder to the pons, while efferent connections extend from the pons to the bladder by way of the pelvic nerve (parasympathetic) and/or the hypogastric nerve (sympathetic). Somatic control over voiding is mediated by the pudendal nerve, which innervates the external bladder sphincter and controls voluntary sphincter contraction and relaxation.

While bladder activity is easy to take for granted, it is an essential part of normal human physiology. Normal adults generally urinate around 6 or 7 times a day, typically during waking hours, though the frequency and timing of voiding can vary significantly between individuals. Overactive bladder (“OAB”) is a condition in which voiding rhythm is disrupted, which is characterized by four symptoms: first, increased urgency to urinate, defined formally as a sudden, compelling desire to urinate that is difficult to deter; second, abnormal urinary frequency, defined as urination more than eight times per day; third, interruption of normal sleep by the urge to void, referred to as “nocturia;” and fourth, “urge incontinence” or involuntary voiding of the bladder during periods of urinary urgency. In the United States, OAB affects an estimated 16% of adults, and about 6% of adults suffer from OAB characterized by urge incontinence. (See Stewart W F, et al. Prevalence and burden of overactive bladder in the United States. World J Urol. May 2003; 20(6):327-36).

OAB has a variety of potential causes which are generally classified as myogenic (arising in the smooth muscle of the bladder), neuropathic (arising from the nervous system), mixed, or idiopathic (lacking a clear etiology). Notwithstanding these categorizations, electrical changes including increased spontaneous contractility and greater electrical coupling between myocytes are observed in detrusor muscle samples taken from patients with both neuropathic and non-neuropathic OAB.

Current treatments for OAB include behavioral therapy to include control over urgency and/or to improve bladder capacity; pharmacotherapy with anticholinergic drugs (e.g. darifenacin, fesoterdione, oxybutynin, etc.) or neurotoxins (e.g. onabotulinumtoxin-A); and electrical neuromodulation of the sacral nerve (for instance, using the InterStim® neuromodulator device (Medtronic, Inc. Minneapolis, Minn.)). While these interventions may be effective to treat OAB in some patients, current pharmacotherapies require repeated administration, while both pharmacological and neuromodulation approaches offer systemic, rather than targeted, intervention, and are accompanied by an increased risk of side effects.

The limitations of current OAB treatments could be addressed by more targeted interventions, and ideally by interventions that specifically target localized bladder abnormalities. However, such therapies would require means by which to identify such abnormalities, direct interventional tools to those abnormalities and, ideally, to verify that therapy has been accurately delivered to them. While cystoscopy is used in a variety of treatments, the relatively large-diameter cystoscope has the potential to cause urethral irritation.

### SUMMARY OF THE DISCLOSURE

The present disclosure, in its various aspects, provides systems, devices and methods for spatially locating abnormalities within the bladder and/or generating virtual maps of the inner surface of the bladder and particularly of the interface between the device and the bladder wall. These aspects may facilitate targeted interventions for conditions such as OAB. In contrast to the systemic interventions currently used to treat OAB, the aspects of the present disclosure are minimally invasive and offer a reduced risk of side effects.

In one aspect, the present disclosure relates to a system for treating a patient, which includes a catheter having an expandable element moveable between a collapsed configuration characterized by a first diameter less than an inner diameter of the urethra of the patient and a second diameter larger than the first diameter. The expandable element includes a plurality of electrodes and at least one sensor for detecting one of a curvature of a portion of the expandable element and a force (or pressure) applied to a portion of the expandable element. The system also preferably includes a controller that is able to perform at least one of the following functions: a) measuring an impedance of at least one of the plurality of electrodes (b) measuring a curvature of the expandable element, (c) measuring a temperature of the expandable element; and (d) delivering an electrical stimulus to the patient via at least one of the plurality of electrodes. The controller is, optionally or additionally, able to compare an impedance measured by a first electrode to one of a pre-determined reference impedance and an impedance measured simultaneously by a second electrode, and based on the comparison, determine whether a portion of the expandable element is apposed to a tissue surface. In some cases, the expandable element includes a plurality of optical fibers, each of which in turn includes a plurality of fiber Bragg gratings. Where such fiber Bragg gratings are used, the optional controller may also be programmed to compare a reflected wavelength from a first optical fiber to one of a predetermined reference wavelength and a reflected wavelength from a second optical fiber and, based on the comparison, determine whether a portion of the expandable element comprising the first optical fiber is in apposition with a tissue surface. In some cases the tip of the catheter is steerable, and in some cases the catheter includes at least one fiber optic imaging elements for transmitting light into a body of a patient and/or transmitting light from the bladder to a detector (such as a camera). In some cases, the expandable element may be a basket comprising a plurality of elongate elements; in others, the expandable element may be a balloon or a helical element. The electrodes are optionally formed from a flexible printed circuit, and/or configured to measure an impedance and deliver a current or voltage. In some cases, each of the plurality of electrodes may be configured to record an electrical activity within the body of a patient and the controller may be further programmed to output an electromyogram and/or to deliver one of an ablative stimulus and a pacing stimulus to a tissue of a patient. Alternatively or additionally, the controller may be configured to receive an electrical signal from a first electrode and, based on the signal, deliver a current through a second electrode or modify an amount of current being delivered through the second electrode. Systems according to this aspect of the disclosure are particularly useful in the diagnosis and treatment of overactive bladder.

In another aspect, the present disclosure relates to a method of treating a patient that includes inserting a steerable catheter into the bladder of the patient; the steerable catheter, as above, includes an expandable element moveable between a collapsed configuration characterized by a first diameter and an expanded configuration characterized by a second diameter larger than the first diameter, which expandable element includes a plurality of electrodes and at least one sensor for detecting one of a curvature of the expandable element and a force applied to the expandable element. The catheter may be used to map a wall of the bladder, which optionally includes expanding the expandable element and detecting apposition between the expandable element and an inner surface of the bladder. Apposition can be detected in multiple ways: for example, the expandable element optionally includes a plurality of optical fibers, each of which includes multiple fiber Bragg gratings as described above. In this case, apposition may be detected by detecting a difference between the curvature of a first optical fiber as indicated by a first wavelength sensed by a first sensor optically communicating with the first optical fiber and one of a predetermined reference curvature and a curvature of a second optical fiber as indicated by a second wavelength sensed by a second sensor optically communicating with the second optical fiber. Alternatively or additionally, apposition may be detected by comparing an impedance measured by a first electrode on the expandable element to one of a predetermined reference impedance and an impedance measured simultaneously by a second electrode on the expandable element. The method also may include delivering an electrical stimulus (e.g. an ablation stimulus, inhibiting stimulus, or pacing stimulus) to a portion of the bladder based on the mapping step.

In yet another aspect, the present disclosure relates to a bladder mapping catheter which includes an expandable element moveable between a collapsed configuration characterized by a first diameter and an expanded configuration characterized by a second diameter larger than the first diameter, which expandable element includes a plurality of electrodes and at least one sensor for detecting one of a curvature of the expandable element and a force applied to the expandable element. The expandable element optionally includes a plurality of optical fibers, each optical fiber comprising a plurality of fiber Bragg gratings. In some cases, each of the plurality of electrodes includes a flexible printed circuit. Each of the electrodes may be, optionally or additionally, configured to deliver electrical stimulus and to receive an electrical signal. In some cases, the expandable element may be a basket, though in other cases the expandable element may be a balloon or a helical structure as described above.

### DRAWINGS

Aspects of the disclosure are described in greater detail below with reference to the following drawings in which like numerals reference like elements, and wherein:

FIG. 1A is a photograph of a cardiac electrophysiological mapping catheter comprising a basket and an electrode array.

FIG. 1B is a schematic depiction of a mapping catheter according to certain embodiments of the present disclosure.

Unless otherwise provided in the following specification, the drawings are not necessarily to scale, with emphasis being placed on illustration of the principles of the disclosure.

### DETAILED DESCRIPTION OF THE PREFERRED EMBODIMENTS

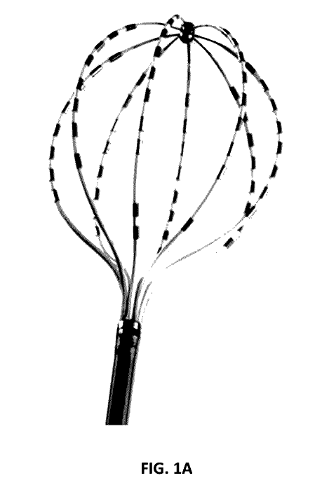

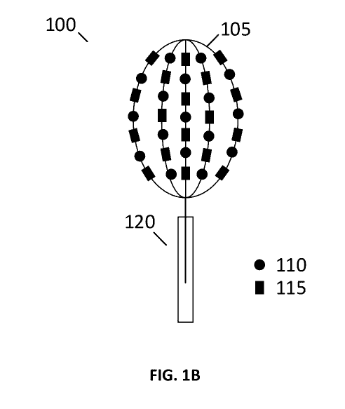

Systems, devices and methods for mapping electrical activity and/or other features of bladder anatomy are provided. Preferred embodiments of the present disclosure utilize an electrode array 100 that can be collapsed and expanded, for example by means of an expandable basket structure 105 (FIG. 1B) or a balloon (not shown) or expandable helical structure (not shown), that includes a plurality of electrodes 110 that are, ideally, operable to both deliver electrical stimulus and record electrical signals (whether endogenous or generated by other electrodes within the array) and which are most preferably operable independently of one another. FIG. 1A shows a cardiac mapping catheter (Constellation™, marketed by Boston Scientific, Marlborough, Massachusetts) that shares certain features with mapping devices of the present disclosure, including that shown in FIG. 1B, such as an expandable basket structure 105 comprising a plurality of electrodes 110, which are regularly spaced along the length and circumference of the expandable structure 105. In addition to these features, in some instances, mapping catheter 100 optionally includes a plurality of sensors 115 useful in detecting apposition of the catheter 100, and particularly the electrode or electrodes 110 with the bladder wall, as illustrated in FIG. 1B.

The sensors 115 are, in some cases, configured to measure curvature of the expandable structure 105, while in other cases, the sensors measure a force (e.g. mechanical, fluid-flow, electrical, etc.) applied by to the expandable structure 105, e.g. by the bladder wall. The sensors can be, in various cases, electrical in nature, e.g. dedicated impedance sensors, can be microfluidic or can be optical. In preferred embodiments, the expandable element 105 comprises a plurality of optical fibers, each fiber comprising a series of fiber Bragg gratings for use as deformation sensors 115. The catheter 100 is connectable to one or more light sources for illuminating each fiber separately, and preferably connects further to one or more photodetection elements capable of detecting light of multiple wavelengths. The principal wavelength λ of light reflected by each Bragg grating within the fiber varies with the degree of curvature of the grating, and in preferred embodiments, mapping systems of the present disclosure include controllers configured to implement an algorithm that takes as inputs one or more of intensity and wavelength emitted by the light source, the intensity and wavelength of light reflected by the Bragg gratings, and the photo-elastic coefficient of the fibers (P_{c}) utilized in the spline, and provides as output a readout of apposition between the spline of the expandable body 105 and a tissue. The measurement of curvature may be done, for example, according to the method of Yi, et al. (“An Orthogonal Curvature Fiber Bragg Grating Sensor Array for Shape Reconstruction,” in Life System Modeling and Intelligent Computing, Communications In Computer and Information Science, Vol. 97, 2010 Springer-Verlag Berlin Heidelberg, which is incorporated by reference herein for all purposes). According to Yi et al., when strain is applied to a fiber Bragg grating strain, the reflected wavelength shifts according to the equation 1 below, in which λ_{B} is the reflected wavelength and ε is the applied strain:

Δλ_{B}=λ_{B}·(1−P_{c})·ε [1]

Thus, the shift in λ_{B} can be used to calculate strain, which in turn can be used to calculate the curvature of the spline using any suitable model of strain and curvature that is appropriate; alternatively, once the expandable member 105 is fully expanded, the curvature of any spline would not be expected to change unless the spline were to contact the bladder wall, so a shift in wavelength may be sufficient in some cases to identify apposition between the spline and the bladder wall. In preferred embodiments, each spline includes multiple fiber Bragg gratings, and these gratings are optionally tuned to reflect different wavelengths of light. Alternatively or additionally, the fiber Bragg gratings may have the same wavelength tuning, and differences in reflected wavelength may be achieved mechanically, for instance by positioning the gratings within portions of the spline having different curvatures, or within spline segments with different photoelastic coefficients P_{c}.

The curvature of individual splines within the expandable element 105 are optionally compared in order to identify which portions of the expandable element 105 are in contact with the bladder wall and which are not. These measurements are also optionally supplemented with direct pressure information from one or more pressure sensors disposed on the spline.

Alternatively or additionally, apposition between portions of the expandable element 105 and the bladder wall can be determined by impedance measurements at each of the electrodes 110 within the array.

The mapping catheter 100 also optionally includes other sensors, such as a temperature sensor that can be used to provide feedback during ablation, accelerometer(s) and/or electromagnetic location sensing elements to provide information on the position and movement of the expandable element 105 within the bladder and/or to provide information on the degree of expansion of the expandable element 105. Each of these sensors, while borne on the catheter 100, may be located in any suitable position, including on or in the catheter shaft, or on or in one or more splines of the expandable element 105. Information about expansion of the element 105 using the above sensing elements together with interpretation of wavelengths reflected from fiber Bragg gratings is particularly useful for determining the location and shape (and thereby forming a virtual map) of the expandable element (in particular the location and shape of the individual splines) in a situation when direct optical visualization is not available.

Additionally, the catheter 100 is optionally designed to rotate (e.g. comprises a coiled or braided layer to transmit torque between the proximal and distal ends of the catheter 100) and to be steered (e.g. by means of one or more wires that can be pushed or pulled to generate curvature at or near the tip, or by means of a steerable sleeve through which catheter 100 is inserted into the bladder). Catheters incorporating these features may be easier to position in close apposition with the bladder wall than catheters without them.

The catheter 100 also optionally includes one or more fiber optic or electronic (camera/led) elements to form a light path to the distal tip of the catheter and/or an imaging path from the distal tip, making it possible to image the bladder directly through the catheter 100 in lieu of or addition to cystoscopic or fluoroscopic bladder imaging (advantageously reducing irritation and attendant electrical noise). Alternatively or additionally, the catheter 100 includes one or more oxygen-sensing elements configured to notify a user when the expandable element is disposed near a region with relatively high oxygen content, signaling that the region is well vascularized; to avoid the risk of hemorrhage, preferred embodiments of the present disclosure do not include ablation or inhibition of regions that are well vascularized.

The mapping catheters described above are typically used as part of a bladder treatment system. First, a mapping catheter 100 is delivered to the bladder through the lumen 120 of a working channel of a cystoscope or, more preferably, through a urinary (i.e. urethral) catheter. The catheter 100 is also connectable to, or includes, a handle element comprising actuators for expanding and contracting the expandable element 105 and for steering the tip of the catheter 100, and includes leads connectable to a waveform generator for delivering electrical stimulus through the electrodes 110 and/or to an amplifier and/or other system for measuring current, voltage, impedance, etc. from the electrodes 110 and, optionally, accelerometer data, curvature information and temperature data. Electrodes may be used to measure point impedance or electromyogram, or they may be used in pairs (such pairs utilizing various combinations of electrodes on the same spline or different splines) with an algorithm to determine the shape and volume of the bladder filled with saline. Furthermore, the impedance and impedance planimetry data may be used with an algorithm to display a virtual photo of the bladder with the device inserted.

With respect to impedance planimetry, in one exemplary protocol, current is delivered using a pair of electrodes and the corresponding voltage is measured using two or more other electrodes within the array; voltage data is processed in view of the relatively low resistivity of urine and saline (roughly 100 Ohms/cm) compared to the relatively higher resistivity of bladder tissue (roughly 800-1000 Ohms/cm), thereby allowing the system to determine which electrodes contact tissue and which are within the bladder volume. A more detailed explanation of impedance planimetry is provided in Lenglinger, “Impedance Planimetry,” in Dysphagia: Diagnosis and Treatment, pp 329-337 (2012, Springer Berlin), which is incorporated by reference herein for all purposes.

In use, the catheter 100 is inserted into the bladder filled with normal saline at a volume lower than the threshold volume of the bladder (i.e. volume at which bladder empties during a concerted contraction), preferably through a lumen of a catheter extending from the urethra into the bladder, and the expandable element 105 is expanded. The catheter 100 is then preferably steered toward the bladder wall guided by impedance measurements from the electrodes 110 and, optionally, by imaging using a cystoscope, fluoroscope, or by a camera element within the catheter 100 itself, which camera can capture light transmitted through the fiber optic splines within the expandable element 105 and thereby provide image data for guiding the catheter 100. Once close apposition between the expandable element 105 and the bladder wall is established, electromyographic recordings are taken using the electrodes 110 at one or more points along the bladder wall to identify a site or sites of aberrant electrical activity. Electrical mapping data generated using the electrodes 110 is optionally superimposed upon, or combined with, other spatial information or mapping data obtained prior to or during the mapping procedure. Sources of this data can include CT scanning, MRI imaging, fluoroscopy, optical imaging using a cystoscope or using optical elements optionally included in the catheter 100; information regarding catheter position obtained from optional accelerometers, gyroscope elements, etc. may useful for accurately merging electrical mapping data with other mapping data, but is not necessarily required.

Once a site or sites of aberrant activity are identified, catheter 100 can be used to deliver electrical stimulus to the site, to ablate or inhibit those sites. For instance, electrodes 110 in close apposition (i.e. contacting, or within a distance of 0-1000 microns) to the bladder wall at the site of aberrant activity can be activated to supply ablation (e.g. radiofrequency) or non-ablative inhibitory stimulus to the bladder wall; the delivery of stimulus can be according to a predetermined program, and/or can vary based upon feedback from catheter elements such as the optional temperature sensor(s) or based on impedance measurements at and around the site where stimulus is being delivered. Those of skill in the art will appreciate that, in other settings, radiofrequency-based thermal ablation of target tissues is associated with a rapid drop in impedance that is believed to correspond with the disruption of cellular structures within the ablation region. In bladder applications, a drop of 20-30% or more in measured impedance is indicative of (though not necessarily definitive of) a complete ablation; similarly, achievement of a target temperature on the electrodes 110 may be integrated into the expandable element 105 using any suitable means, including without limitation adhesives. In some cases, the electrodes include flexible, printed circuits.

The various aspects of the present disclosure described above may offer several advantages over currently used OAB treatments, including providing long-lasting local treatment of aberrant electrical activity underlying OAB without affecting other tissues in the same way as systemically administered pharmacotherapies or electrical interventions targeting the spinal cord and/or nerves that innervate the bladder and adjacent structures. In addition, certain features of the present disclosure may facilitate its use in doctors' offices, without the need for fluoroscopic or other real-time imaging, potentially reducing procedure costs, and may include multiple safety mechanisms to prevent, for example, ablation of highly-vascularized bladder regions.

### CONCLUSION

The foregoing examples have focused on mapping and ablation of regions of the bladder to limit aberrant electrical activity and, thereby, to treat OAB. Those of skill in the art, however, will understand that the embodiments illustrated above are useful in the treatment of a variety of conditions related to aberrant spontaneous electrical activity in bodily organs or lumens. For instance, electrodes and systems similar to those described above may be useful in treating conditions of the digestive tract, including without limitation the stomach and/or the large and small intestines. The use of the electrodes, devices, systems and methods described above to treat such conditions are within the scope of the present disclosure.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present disclosure have described above. It is, however, expressly noted that the present disclosure is not limited to those embodiments, but rather the intention is that additions and modifications to what was expressly described herein are also included within the scope of the disclosure. Moreover, it is to be understood that the features of the various embodiments described herein were not mutually exclusive and can exist in various combinations and permutations, even if such combinations or permutations were not made express herein, without departing from the spirit and scope of the disclosure. In fact, variations, modifications, and other implementations of what was described herein will occur to those of ordinary skill in the art without departing from the spirit and the scope of the disclosure. As such, the disclosure is not to be defined only by the preceding illustrative description.

1. A system for treating a patient, comprising:
a steerable catheter, comprising an expandable element moveable between a collapsed configuration characterized by a first diameter and an expanded configuration characterized by a second diameter larger than the first diameter, the expandable element comprising a plurality of electrodes and at least one sensor for detecting at least one of a curvature of the expandable element and a force applied to the expandable element.

2. The system according to claim 1, further comprising a controller configured to perform at least one function selected from the group consisting of (a) measuring an impedance (b) measuring a curvature of the expandable element, (c) measuring a temperature of the expandable element; and (d) delivering an electrical stimulus to at least one of the plurality of electrodes.

3. The system according to claim 2, wherein the controller is configured to compare an impedance measured by a first electrode to one of a pre-determined reference impedance and an impedance measured simultaneously by a second electrode and, based on the comparison, determine whether a portion of the expandable element is apposed to a bladder wall.

4. The system according to claim 2, wherein the expandable element includes a plurality of optical fibers, each optical fiber comprising a plurality of fiber Bragg gratings, and the controller is configured to receive wavelength information from each of the plurality of optical fibers and determine a curvature of each of the plurality of optical fibers.

5. The system according to claim 4, wherein the controller is configured to indicate to a user that a portion of the expandable element is in apposition with a tissue surface based on a curvature of at least one of the plurality of optical fibers.

6. The system according to claim 2, wherein the catheter includes at least one fiber optic imaging element for transmitting light into a bladder of a patient, the controller being configured to output an image of the bladder of the patient to a display.

7. The system according to claim 2, wherein each of the plurality of electrodes is configured to measure an impedance and to deliver a current.

8. The system according to claim 2, wherein each of the plurality of electrodes is configured to record an electrical activity within the bladder of a patient and the controller is further programmed to output an electromyogram.

9. The system according to claim 2, wherein each of the plurality of electrodes is configured to deliver one of an ablative stimulus and a pacing stimulus to a bladder of a patient.

10. The system according to claim 2, wherein the controller is configured to receive an electrical signal from a first electrode and, based on the signal, deliver a current through a second electrode or modify an amount of current being delivered through the second electrode

11. A method of treating a patient, comprising the steps of:
inserting, into the bladder of the patient, a steerable catheter, comprising:
an expandable element moveable between a collapsed configuration characterized by a first diameter and an expanded configuration characterized by a second diameter larger than the first diameter, the expandable element comprising a plurality of electrodes and at least one sensor for detecting at least one of a curvature of the expandable element and a force applied to the expandable element; and
mapping, with the expandable element, a wall of the bladder.

12. The method of claim 11, wherein the step of mapping an inner surface of the bladder includes:
expanding the expandable element; and
detecting apposition between the expandable element and an inner surface of the bladder.

13. The method of claim 12, wherein the expandable element includes a plurality of optical fibers, each optical fiber including one or more fiber Bragg gratings, and the step of detecting apposition between the expandable element and the inner surface of the bladder includes detecting a difference between the curvature of a first optical fiber as indicated by a first wavelength sensed by a first sensor optically communicating with the first optical fiber and one of a predetermined reference curvature and a curvature of a second optical fiber as indicated by a second wavelength sensed by a second sensor optically communicating with the second optical fiber.

14. The method of claim 12, wherein the step of detecting apposition between the expandable element and the inner surface of the bladder includes comparing an impedance measured by a first electrode on the expandable element to one of a predetermined reference impedance and an impedance measured simultaneously by a second electrode on the expandable element.

15. The method of claim 12, further comprising the step of delivering an electrical stimulus to a portion of the bladder based on the mapping step.

16. A bladder mapping catheter, comprising:
an expandable element moveable between a collapsed configuration characterized by a first diameter and an expanded configuration characterized by a second diameter larger than the first diameter, the expandable element comprising a plurality of electrodes and at least one sensor for detecting at least one of a curvature of the expandable element and a force applied to the expandable element.

17. The mapping catheter of claim 16, wherein the expandable element includes a plurality of optical fibers, each optical fiber comprising a plurality of fiber Bragg gratings.

18. The mapping catheter of claim 16, wherein each of the plurality of electrodes includes a flexible printed circuit.

19. The mapping catheter of claim 16, wherein each of the plurality of electrodes is configured to deliver electrical stimulus and to receive an electrical signal.

20. The mapping catheter of claim 16, wherein the expandable element is a basket.
